(19) United States
(12) Patent Application Publication
(10) Pub. No.: US 2022/0133788 A1
(43) Pub. Date: May 5, 2022

(54) BAFF THERAPY TO PROMOTE ANTI-TUMOR IMMUNITY

(71) Applicant: The Johns Hopkins University, Baltimore, MD (US)

(72) Inventors: Elizabeth A. Jaffee, Lutherville, MD (US); Mark Yarchoan, Baltimore, MD (US)

(21) Appl. No.: 16/956,537

(22) PCT Filed: Dec. 19, 2018
(86) PCT No.: PCT/US18/66566

§ 371 Date: Jun. 19, 2020

Related U.S. Application Data
(60) Provisional application No. 62/607,584, filed on Dec. 19, 2017.

(51) Int. Cl. A61K 35/17; A61K 35/13; A61K 39/00; A61K 38/19; A61K 35/28

(52) U.S. Cl. CPC A61K 35/17; A61K 35/13; A61K 39/0011; A61K 2039/55522; A61K 35/28; A61K 2039/5152; A61K 38/191

(57) ABSTRACT

Anti-tumor immune response are generated by induction of activated B cells to provide costimulatory signals necessary for T cell activation. Certain compositions are combined with anti-immune checkpoint inhibitors to generate a synergistic anti-tumor response.

### CROSS-REFERENCE TO RELATED APPLICATIONS

This application claims the benefit of priority under 35 U.S.C. § 119(e) to U.S. Provisional Application No. 62/607,584, filed on Dec. 19, 2017, which is incorporated herein by reference in its entirety.

### FIELD OF THE INVENTION

Embodiments of the invention are directed to induction of activated B cells to provide costimulatory signals necessary for T cell activation, in combination with anti-immune checkpoint inhibitors to generate a synergistic anti-tumor response.

### BACKGROUND

B-cell activating factor (BAFF) is a cytokine that belongs to the tumor necrosis factor (TNF) ligand superfamily and acts as a ligand for receptors BAFF-R (BR3), TACI (transmembrane activator and calcium modulator and cyclophilin ligand interactor) and BCMA (B-cell maturation antigen). The interaction between BAFF and its receptors triggers signals essential for the formation and maintenance of B cells, which in turn synthesizes immunoglobulins in response to invasion by a foreign substance. Appropriate levels of BAFF in a patient help maintain normal levels of immunity whereas inadequate levels can lead to immunodeficiency and excessive levels can result in abnormally high antibody production.

BAFF can exist in three forms: membrane bound (mbBAFF), soluble trimeric BAFF (sBAFF) and a multimeric form consisting of 60 BAFF monomers (BAFF 60mer). The relative importance of the various forms of BAFF in normal and disease physiology is not well understood. As noted, BAFF binds to three receptors, BAFFR (BR3), TACI and BCMA. A proliferation-inducing ligand (APRIL), a related member of the TNF receptor ligand family, has been shown to bind with high affinity to TACI and BCMA. In contrast to the high affinity APRIL:BCMA interaction, the BAFF:BCMA interaction is of low affinity (1-2 μM) and is not believed to be play an important role in vivo.

BAFF co-stimulates the proliferation of B cells in the presence of anti-IgM (Schneider et al., J. Exp. Med. 189:1747-1756 (1999)) and is able to signal through three receptors: BCMA (B cell maturation antigen), TACI (transmembrane activator and cyclophilin ligand interactor, and BR3 (BAFF receptor 3, also known as BAFF-R). Fusion proteins of these receptors with the CH1, CH2, and hinge region of human IgG1 block the proliferation of B cells induced by BAFF. (Gross et al., Nature 404:995-999 (2000); Gross et al., Immunity 15: 289-302 (2001); Thompson et al., J. Exp. Med. 192:129-135 (2000); Thompson et al., Science 293:2108-2111 (2001)).

Soluble BAFF is expressed at high levels in individuals with systemic lupus erythematosus (SLE) and in inflamed target organs such as the kidney. Soluble BAFF serves as a critical factor for B cell homeostasis and survival. Autoantibody formation by BAFF-dependent B cells results in glomerular IC deposits, initially at the glomerular basement membrane (GBM), mesangium and interstitial tissue within the proximal tubular epithelial cells (PTEC). These IC deposits lead to complement fixation and neutrophil activation resulting in local kidney damage. Inflammatory mediators (e.g. IL6, IL8, MCP-1) produced by the damaged kidney cells (MC, PTEC, renal fibroblasts, endothelial cells) fuel an inflammatory cycle by increasing immune cell infiltration (e.g. B cells, T cells, dendritic cells, neutrophils and macrophages).

BCMA and TACI bind to both BAFF and APRIL. Gross et al., Nature 404:995-999 (2000); Wu et al., J. Biol. Chem. 275:35478-35485 (2000); Xia et al., J. Exp. Med. 192:137-143 (2000); Yan et al., Nat. Immunol. 1:37-41 (2000); Yu et al., Nat. Immunol. 1:252-256 (2000). BR3 is expressed in all peripheral B cells and is specific for BAFF, i.e., unlike BCMA and TACI, BR3 does not bind APRIL. Mice lacking BR3 have a similar phenotype to BAFF knockout mice. Thompson et al., Science 293:2108-2111 (2001); Yan et al., Curr. Biol. 11:1547-1552 (2001). Recently, studies with monomeric receptors have shown that BAFF binds BR3 with 100-fold higher affinity than it binds BCMA. Rennert et al., J. Exp. Med. 192:1677-1684 (2000); Patel et al., J. Biol. Chem. 279:16727-16735 (2004); Day et al., Biochemistry 44:1919-1931 (2005).

### SUMMARY

Embodiments of the invention are directed to compositions which provide stimulatory signals for T cell activation, proliferation and survival. Compositions include B-cell activating factor (BAFF), BAFF and checkpoint inhibitors in the treatment of cancers.

In certain embodiments, a method of treating cancer comprises administering to a subject in need thereof, a composition comprising a therapeutically effective amount of B-cell activating factor (BAFF). In certain embodiments, BAFF is administered as an adjuvant to increase a specific immune response. Examples include an anti-tumor specific immune response, vaccines and the like.

In certain embodiments, a method of treating cancer comprises administering to a subject in need thereof, a composition comprising a therapeutically effective amount of B-cell activating factor (BAFF) and at least one checkpoint inhibitor. In certain embodiments, the checkpoint inhibitor comprises an inhibitor of: PD-1, PD-L1, PD-L2, CTLA4, TIM-3, LAG-3, CEACAM-1, CEACAM-5, VISTA, BTLA, TIGIT, LAIR1, CD 160, 2B4 or TGFR-β.

In certain embodiments, the at least one checkpoint inhibitor is anti-PD-1.

In certain embodiments, the BAFF increases B-cell expression of CD40 and/or CD86 as compared to a control. In certain embodiments, the BAFF-treated B cells stimulate CD4⁺ T cell activity. In certain embodiments, the BAFF increases reduces T regulatory cell activity, amounts or function and increases TH17 activity, amounts or function.

In certain embodiments, the BAFF is a polynucleotide or oligonucleotides thereof, a protein or active fragments thereof, a cell expressing BAFF, a cell comprising a vector encoding BAFF, a vector encoding BAFF or active fragments thereof.

In certain embodiments, a method of treating cancer, comprises co-culturing ex vivo, CD4⁺ T cells with B-cell activating factor (BAFF)-treated B cells obtained from a subject suffering from cancer; re-infusing the CD4⁺ T cells and BAFF-treated B cells into the subject. In certain embodiments, the B cells are cultured with B-cell activating factor (BAFF). In certain embodiments, the CD4⁺ T cells and BAFF-treated B cells are co-cultured with tumor cells obtained from the subject. In other embodiments, the CD4⁺ T cells and BAFF-treated B cells are co-cultured with tumor cell antigens. In further embodiments, the CD4⁺ T cells and BAFF-treated B cells are co-cultured with at least one checkpoint inhibitor. In other embodiments, the at least one checkpoint inhibitor is co-administered to the patient, during re-infusion of the CD4⁺ T cells and BAFF-treated B cells. In other embodiments, the at least one checkpoint inhibitor is administered to the patient prior to, during and/or after re-infusion of the CD4⁺ T cells and BAFF-treated B cells.

In certain embodiments, an isolated cell comprises a vector encoding a B-cell activating factor (BAFF). In other embodiments, the cell is a mammalian cell. In certain embodiments, the cell is a B cell, a stem cell, a bone marrow cell, or cell-line.

In other embodiments, a method of increasing an antigen specific immune response in vivo, comprises isolating B cells from a subject; culturing the B cells with an antigen and B-cell activating factor (BAFF); co-culturing the BAFF- and antigen culture B cells with CD4⁺ T cells and reinfusing the T and B cells into the subject. The antigen can be any desired antigen, e.g. a tumor antigen, viral antigen and the like. The method may include administering a checkpoint inhibitor. Examples of checkpoint inhibitor include, without limitation an inhibitor of: PD-1, PD-L1, PD-L2, CTLA4, TIM-3, LAG-3, CEACAM-1, CEACAM-5, VISTA, BTLA, TIGIT, LAIR1, CD 160, 2B4 or TGFR-β.

In other embodiments, a method of inducing an anti-tumor response in vivo, comprises isolating B cells from a subject; transforming the B cells comprising a vector encoding B-cell activating factor (BAFF); pulsing or culturing the B cells with a tumor antigen; and re-infusing the cells into the patient. In certain embodiments, a checkpoint inhibitor is administered.

In certain embodiments, the B cells or cells comprising a vector encoding B-cell activating factor (BAFF) are cultured with tumor antigens, tumor cells and/or antigen presenting cells. In other embodiments, the CD4⁺ T cells, BAFF-treated B cells or cells comprising a vector encoding B-cell activating factor (BAFF) are cultured with tumor antigens, tumor cells and/or antigen presenting cells.

Other aspects are described infra.

### Definitions

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In preferred embodiments, the genes, nucleic acid sequences, amino acid sequences, peptides, polypeptides and proteins are human.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. In some embodiments, such cells exhibit such characteristics in part or in full due to the expression and activity of immune checkpoint inhibitors, such as PD-1, PD-L1, PD-L2, and/or CTLA-4. Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The term “checkpoint inhibitor” means a group of molecules on the cell surface of CD4⁺ and/or CD8⁺ T cells that fine-tune immune responses by down-modulating or inhibiting an anti-tumor immune response. Immune checkpoint proteins are well known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR (see, for example, WO 2012/177624). “Anti-immune checkpoint inhibitor therapy” refers to the use of agents that inhibit immune checkpoint inhibitors. Inhibition of one or more immune checkpoint inhibitors can block or otherwise neutralize inhibitory signaling to thereby upregulate an immune response in order to more efficaciously treat cancer. Exemplary agents useful for inhibiting immune checkpoint inhibitors include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint inhibitor nucleic acids, or fragments thereof. Exemplary agents for upregulating an immune response include antibodies against one or more immune checkpoint inhibitor proteins block the interaction between the proteins and its natural receptor(s); a non-activating form of one or more immune checkpoint inhibitor proteins (e.g., a dominant negative polypeptide); small molecules or peptides that block the interaction between one or more immune checkpoint inhibitor proteins and its natural receptor(s); fusion proteins (e.g. the extracellular portion of an immune checkpoint inhibition protein fused to the Fe portion of an antibody or immunoglobulin) that bind to its natural receptor(s); nucleic acid molecules that block immune checkpoint inhibitor nucleic acid transcription or translation; and the like. Such agents can directly block the interaction between the one or more immune checkpoint inhibitors and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and anti-CTLA-4 antibodies, either alone or used in combination.

The term “co-administer” refers to the simultaneous presence of two active agents in the blood of an individual. Active agents that are co-administered can be concurrently or sequentially delivered.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, the phrase “consisting essentially of” refers to the genera or species of active pharmaceutical agents included in a method or composition, as well as any inactive carrier or excipients for the intended purpose of the methods or compositions.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the invention are not limited to use of a specific cut-point in comparing the level of expression product in the test sample to the control.

As used herein, an “effective amount,” “therapeutically effective amount” or “effective dose” is an amount of a composition (e.g., a therapeutic composition or agent) that produces at least one desired therapeutic effect in a subject, such as preventing or treating a target condition or beneficially alleviating a symptom associated with the condition.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

“Mammal” covers warm blooded mammals that are typically under medical care (e.g., humans and domesticated animals). Examples include feline, canine, equine, bovine, and human, as well as just human.

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats).

The terms “response” or “responsiveness” refers to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

An “RNA interfering agent” as used herein, is defined as any agent which interferes with or inhibits expression of a target gene by RNA interference (RNAi). Such RNA interfering agents include, but are not limited to, nucleic acid molecules including RNA molecules which are homologous to the target gene of the invention, or a fragment thereof, short interfering RNA (siRNA), and small molecules which interfere with or inhibit expression of a target nucleic acid by RNA interference (RNAi). “RNA interference (RNAi)” is an evolutionally conserved process whereby the expression or introduction of RNA of a sequence that is identical or highly similar to a target nucleic acid results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target nucleic acids. As used herein, “inhibition of target nucleic acid expression” or “inhibition of marker gene expression” includes any decrease in expression or protein activity or level of the target nucleic acid or protein encoded by the target nucleic acid. The decrease may be of at least 30%, 40%, 50%, 60%, 70%, 80%, 90%, 95% or 99% or more as compared to the expression of a target nucleic acid or the activity or level of the protein encoded by a target nucleic acid which has not been targeted by an RNA interfering agent.

As used herein, the terms “preventing” and grammatical variants thereof refer to an approach for preventing the development of, or altering the pathology of, a condition, disease or disorder. Accordingly, “prevention” may refer to prophylactic or preventive measures. For the purposes of this invention, beneficial or desired clinical results include, but are not limited to, prevention or slowing of symptoms, progression or development of a disease, whether detectable or undetectable. A subject (e.g., a human) in need of prevention may thus be a subject not yet afflicted with the disease or disorder in question. The term “prevention” includes slowing the onset of disease relative to the absence of treatment, and is not necessarily meant to imply permanent prevention of the relevant disease, disorder or condition. Thus “preventing” or “prevention” of a condition may in certain contexts refer to reducing the risk of developing the condition, or preventing or delaying the development of symptoms associated with the condition.

The term “targeted therapy” refers to administration of agents that selectively interact with a chosen biomolecule to thereby treat cancer, e.g. immunotherapy targeting tumor antigens.

As used herein “TH17” cells are T helper 17 cells which are a subset of pro-inflammatory T helper cells defined by their production of interleukin 17 (IL-17). TH17 cells are developmentally distinct from TH1 and TH2 lineages. TH17 cells play an important role in maintaining mucosal barriers and contributing to pathogen clearance at mucosal surfaces, but they have also been implicated in autoimmune and inflammatory disorders. The loss of TH17 cell populations at mucosal surfaces has been linked to chronic inflammation and microbial translocation.

As used herein, “treating” or “treatment” and grammatical variants thereof refer to an approach for obtaining beneficial or desired clinical results. The term may refer to slowing the onset or rate of development of a condition, disorder or disease, reducing or alleviating symptoms associated with it, generating a complete or partial regression of the condition, or some combination of any of the above. For the purposes of this invention, beneficial or desired clinical results include, but are not limited to, reduction or alleviation of symptoms, diminishment of extent of disease, stabilization (i.e., not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject (e.g., a human) in need of treatment may thus be a subject already afflicted with the disease or disorder in question. The term “treatment” includes inhibition or reduction of an increase in severity of a pathological state or symptoms relative to the absence of treatment, and is not necessarily meant to imply complete cessation of the relevant disease, disorder or condition.

The term “untargeted therapy” refers to administration of agents that do not selectively interact with a chosen biomolecule yet treat cancer. Representative examples of untargeted therapies include, without limitation, chemotherapy, gene therapy, and radiation therapy.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Where any nucleotide sequence or amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

### BRIEF DESCRIPTION OF THE DRAWINGS

FIG. 1 is a series of graphs from flow cytometric analyses showing that B cells treated with BAFF express high levels of PD-L1.

FIG. 2 is a series of graphs from flow cytometric analyses showing B cells treated with BAFF have increased CD86 and CD40 expression.

FIG. 3 is a series of graphs from flow cytometric analyses showing B cells treated with BAFF have an improved ability to stimulate CD4⁺ T cells.

FIG. 4 is a series of graphs showing proliferation of CD4⁺ T cells in response to BAFF-treated B cells.

FIG. 5 is a graph showing recombinant BAFF synergies with anti-PD-1 therapy promotes ant-tumor immunity.

FIGS. 6A, 6B are graphs showing that BAFF is an effective therapeutic anti-cancer vaccine adjuvant.

FIG. 7A, 7B are graphs showing that BAFF as a vaccine adjuvant can increase the number of anti-tumor IgG antibodies.

FIGS. 8A and 8B are plots showing that B cells treated with BAFF show high levels of PD-L1 (FIG. 8A) and MHCII (FIG. 8B) expression. 1×10⁶ splenocytes from a B6 mouse were cultured for 72 hours in a flat bottom well of a 48-well plate containing 400 microliters of CLT medium (RPMI with 10% Fetal bovine serum, 1% L-glutamine, 1% penicillin/streptomycin, and 50 μM β-mercaptoethanol), with or without recombinant mouse BAFF (1 μg). Flow cytometry was performed on the cells after 72 hours. The BAFF-treated cells express high levels of PD-L1 in a dose-dependent fashion.

FIGS. 9A-9D are plots showing that B cells treated with BAFF show increased MHCII (FIGS. 9A and 9C) and CD40 (FIGS. 9B and 9D) expression.

FIG. 10 is a series of plots showing that BAFF-treated B cells have an improved ability to stimulate CD4⁺ T cells. B cells were isolated from a B6 mouse using an EASYSEP™ Human B Cell Enrichment Kit. 0.5×10⁶ B cells per well were cultured in a flat bottom well of a 48-well plate containing 400 microliters of CLT medium (RPMI with 10% Fetal bovine serum, 1% L-glutamine, 1% penicillin/streptomycin, and 50 μM β-mercaptoethanol). Control or recombinant mouse BAFF (3 μg) were added to the well for 24 hours. For the last 8 hours, Ova peptide (sequence SLKISQAVHAAHAEINEAGR, SEQ ID NO: 1) were added. The plate was then spun and washed three times to remove any BAFF or excess peptide not processed by the B cells. B cells were resuspended in 400 microliters of fresh CLT medium. 1×10⁶ CD4 T cells from an OT2 mouse were added to each well containing B cells, allowing 48 hours of coculture with the B cells. CD4 T cells cocultured with BAFF-treated peptide pulsed B cells showed significant increase in CD44 and CD69 expression at the end of the 48 hour coculture experiment.

FIG. 11 is a series of plots showing that BAFF-treated B cells have an improved ability to stimulate CD4⁺ T cells. The experiments from FIG. 10 were repeated using CFSE-stained CD4⁺ T cells to demonstrate CD4⁺ T cell proliferation. These results show that BAFF-treated B cells have increased activation and an improved ability to stimulate and expand CD4⁺ T cells. However, they upregulate PD-L1 as a major compensatory mechanism.

FIG. 12 is a graph showing that systemic recombinant BAFF synergizes with anti-PD-1 therapy to promote anti-tumor immunity.

FIGS. 13A and 13B are plots showing that systemic recombinant BAFF reduces T regulatory cells (FIG. 13B) and increased TH17 (FIG. 13A) cells in tumor bearing mice.

FIGS. 14A and 14B are graphs showing that BAFF is an effective therapeutic anticancer vaccine adjuvant. FIG. 14A compares the tumor volume (mm³) between Mock-Vac and BAFF-Vac. FIG. 14B compares the percent survival between mice receiving the Mock-Vac versus the BAFF-Vac. A 3T3-derived cell line that secretes mouse BAFF. was created. FIGS. 14A and 14B show that use of this bystander BAFF-secreting cell line induces a more potent anti-tumor immune response when coadministered with irradiated tumor cells, as compared to a 3T3-mock cell line which does not secrete BAFF. Here, 20 NeuN mice were administered 5×10⁵ NT2.5 tumor cells (a mouse model of HER2⁺ breast cancer), injected subcutaneously into the R mammillary gland. On day 3 after tumor inoculation, mice received 3×10⁶ irradiated 3T3-BAFF+3×10⁶ irradiated NT2.5 tumor cells (BAFF-Vac, n=10) or 3×10⁶ irradiated 3T3-mock+3×10⁶ irradiated NT2.5 tumor cells (Mock-Vac, n=10), injected over 3 limbs. Survival for the group receiving irradiated 3T3-BAFF+NT2.5 tumor cells was greater than the group receiving the mock vaccine.

FIG. 15 is a series of plots showing that BAFF as a vaccine adjuvant can increase the number of anti-tumor IgG antibodies. Mice were treated as described for FIGS. 14A and 14B. At days 7 and 15 after vaccination with BAFF-Vac or Mock-Vac, 150 μl was collected using tail bleeds from each mouse using heparinized capillary tubes. The collected blood was spun down and serum was collected. Using FACs of the NeuN tumor cells, with the mouse serum used as a primary antibody, and using a secondary anti-IgG antibody, a marked increase in anti-tumor IgG was found at 15 days.

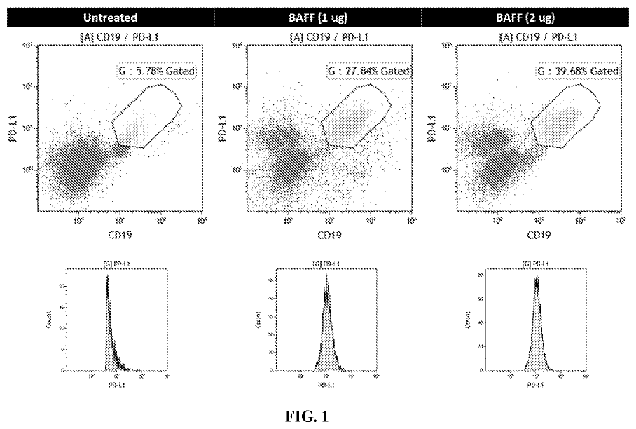

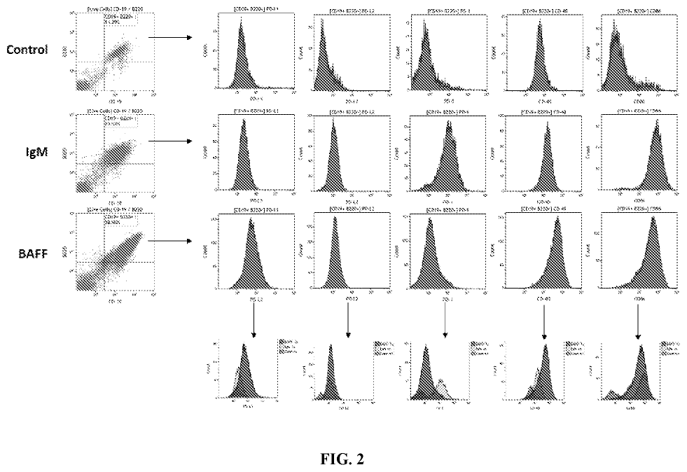

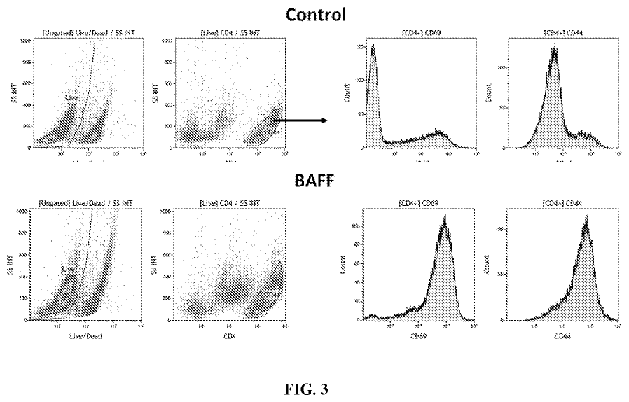

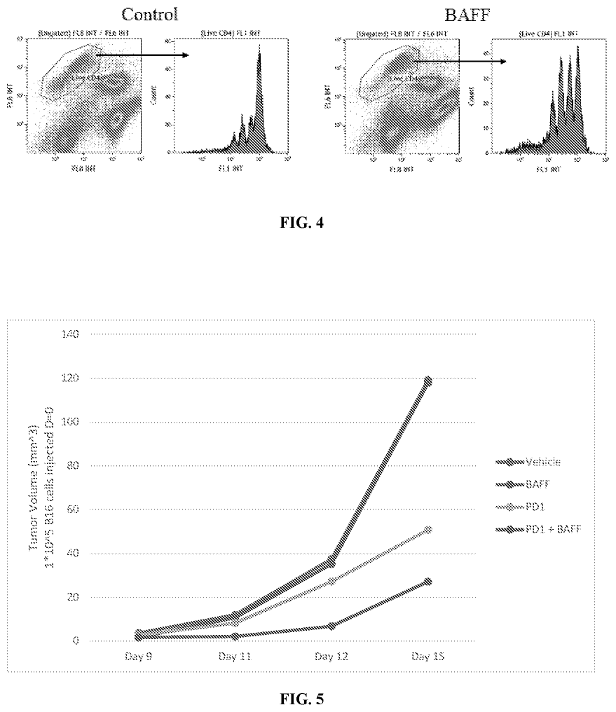

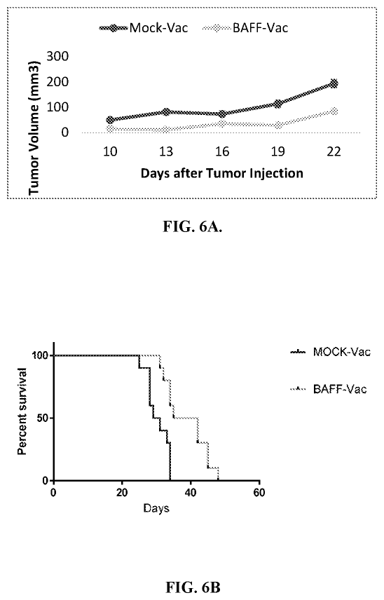

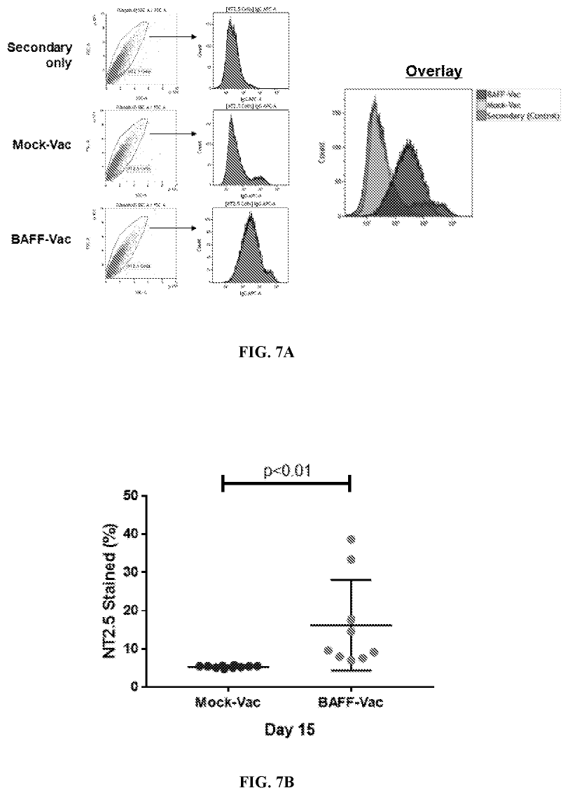

### DETAILED DESCRIPTION

B-cell activating factor (BAFF) also known as tumor necrosis factor ligand superfamily member 13B as well as B Lymphocyte Stimulator (BLyS) is a protein that binds to three known receptors: TNFRSF13B/TACI, TNFRSF17/BCMA, and TNFRSF13C/BAFF-R. BAFF is thought to contribute to the development of autoimmune disorders including lupus.

Recombinant BAFF was shown, in the examples section which follows, to induce activated B cells which provide costimulatory signals necessary for T cell activation, proliferation, and survival. It was also shown that BAFF leads to a compensatory increase of PD-L1 expression on B cells. The combination of BAFF with an anti-PD-1 pathway antibody leads to a synergistic anti-tumor response.

Accordingly, in certain embodiments, a composition comprising a therapeutically effective amount of B-cell activating factor (BAFF). In other embodiments, a method of treating cancer comprises administering to a subject in need thereof, a composition comprising a therapeutically effective amount of B-cell activating factor (BAFF).

The amino acid sequences of naturally occurring full-length human BAFF, BCMA, TACI, and BR3 are available under GenBank™ accession numbers AAD25356, BAB60895, AAP57629, and AAK91826, respectively.

In certain embodiments, a method of treating cancer comprises administering to a subject in need thereof, a composition comprising a therapeutically effective amount of B-cell activating factor (BAFF) and at least one checkpoint inhibitor. In certain embodiments, a checkpoint inhibitor comprises an inhibitor of: PD-1, PD-L1, PD-L2, CTLA4, TIM-3, LAG-3, CEACAM-1, CEACAM-5, VISTA, BTLA, TIGIT, LAIR1, CD 160, 2B4 or TGFR-β.

Compositions and Methods of Treatment

Compositions can include cells expressing BAFF, vectors encoding BAFF molecules, a polynucleotide encoding BAFF or oligonucleotides thereof, a polypeptide of BAFF or peptides thereof, mutants, active fragments, orthologs, analogs or combinations thereof.

In certain embodiments, a composition comprises an isolated cell comprising a vector encoding B-cell activating factor (BAFF) or active fragments thereof. The cells are preferably of mammalian origin, however, the invention is not so limited. In certain embodiments, the cell is a B cell, a stem cell, a bone marrow cell, a tumor cell, or cell-line.

In certain embodiments, a method of treating cancer, comprises co-culturing ex vivo, CD4⁺ T cells with B-cell activating factor (BAFF)-treated B cells obtained from a subject suffering from cancer and re-infusing the CD4⁺ T cells and BAFF-treated B cells into the subject. The CD4⁺ T cells and BAFF-treated B cells can be co-cultured with tumor cells obtained from the subject or tumor antigens, so as to induce tumor cell specific T cells. The tumor cells can be irradiated.

In certain embodiments, a method of treating cancer comprises administering to a subject in need thereof, a composition comprising a therapeutically effective amount of B-cell activating factor (BAFF). In certain embodiments, a method of treating cancer comprises administering to a subject in need thereof, a composition comprising a therapeutically effective amount of B-cell activating factor (BAFF) and an anti-tumor vaccine. Examples of anti-tumor vaccines include irradiated tumor cells, tumor antigens etc.

In other embodiments, a method of increasing an antigen specific immune response in vivo, comprises administering to a subject a composition comprising BAFF and a desired antigen for which a specific immune response is desired. The antigen can be a vaccine, a peptide, an irradiated cell, polynucleotide, oligonucleotide, etc.

In certain embodiments, a composition comprising BAFF is administered as an adjuvant.

In other embodiments, a method of increasing an antigen specific immune response in vivo, comprises isolating B cells from a subject; culturing the B cells with an antigen and B-cell activating factor (BAFF); co-culturing the BAFF- and antigen culture B cells with CD4⁺ T cells and reinfusing the T and B cells into the subject. The antigen can be any desired antigen, e.g. a tumor antigen, viral antigen and the like. The method may include administering a checkpoint inhibitor. Examples of checkpoint inhibitor include, without limitation an inhibitor of: PD-1, PD-L1, PD-L2, CTLA4, TIM-3, LAG-3, CEACAM-1, CEACAM-5, VISTA, BTLA, TIGIT, LAIR1, CD 160, 2B4 or TGFR-β.

In other embodiments, a method of inducing a tumor response in vivo, comprises isolating B cells from a subject; transforming the B cells comprising a vector encoding B-cell activating factor (BAFF); pulsing or culturing the B cells with a tumor antigen; and re-infusing the cells into the patient. In certain embodiments, a checkpoint inhibitor is administered.

In certain embodiments, the B cells or cells comprising a vector encoding B-cell activating factor (BAFF) are cultured with tumor antigens, tumor cells and/or antigen presenting cells. In other embodiments, the CD4⁺ T cells, BAFF-treated B cells or cells comprising a vector encoding B-cell activating factor (BAFF) are cultured with tumor antigens, tumor cells and/or antigen presenting cells.

The compositions embodied herein can be used to increase an antigen specific immune response in vivo. In certain embodiments a method of increasing an antigen specific immune response in vivo, comprises isolating B cells from a subject; culturing the B cells with an antigen and B-cell activating factor (BAFF); co-culturing the BAFF- and antigen culture B cells with CD4⁺ T cells and reinfusing the T and B cells into the subject. The antigen can be any desired antigen for treating a disease, whether a tumor, virus etc.

In certain embodiments, an antigen specific immune response is directed to one or more tumor antigens. Accordingly, in some embodiments, a method of inducing a tumor response in vivo, comprises isolating B cells from a subject; transforming the B cells with a vector encoding B-cell activating factor (BAFF), or, transforming a cell with a vector encoding B-cell activating factor (BAFF); pulsing or culturing the B cells with a tumor antigen or tumor cells and re-infusing the cells into the patient. The tumor cells are irradiated or are non-irradiated. In certain embodiments, the cells are cultured with antigen presenting cells, e.g. macrophages, dendritic cells. In certain embodiments, a checkpoint inhibitor is administered. In certain embodiments, a checkpoint inhibitor comprises an inhibitor of: PD-1, PD-L1, PD-L2, CTLA4, TIM-3, LAG-3, CEACAM-1, CEACAM-5, VISTA, BTLA, TIGIT, LAIR1, CD 160, 2B4 or TGFR-β.

In certain embodiments, a composition comprises an isolated B lymphocyte, an isolated T lymphocyte, a B-cell activating factor (BAFF) or active fragments thereof. The composition optionally comprises tumor antigens or tumor cells. In certain embodiments, the composition further comprises a checkpoint inhibitor, wherein the checkpoint inhibitor comprises an inhibitor of: PD-1, PD-L1, PD-L2, CTLA4, TIM-3, LAG-3, CEACAM-1, CEACAM-5, VISTA, BTLA, TIGIT, LAIR1, CD 160, 2B4 or TGFR-β.

In certain embodiments, one or more tumor antigens include, but is not limited to, 5 alpha reductase, alpha-fetoprotein, AM-1, APC, April, BAGE, beta-catenin, Bcl12, bcr-abl, CA-125, CASP-8/FLICE, Cathepsins, CD19, CD20, CD21, CD23, CD22, CD33 CD35, CD44, CD45, CD46, CD5, CD52, CD55, CD59, CDCl27, CDK4, CEA, c-myc, Cox-2, DCC, DcR3, E6/E7, CGFR, EMBP, Dna78, farnesyl transferase, FGF8b, FGF8a, FLK-1/KDR, folic acid receptor, G250, GAGE-family, gastrin 17, gastrin-releasing hormone, GD2/GD3/GM2, GnRH, GnTV, GP1, gp100/Pme117, gp-100-in4, gp15, gp75/TRP-1, hCG, heparanse, Her2/neu, HMTV, Hsp70, hTERT, IGFR1, IL-13R, iNOS, Ki67, KIAA0205, K-ras, H-ras, N-ras, KSA, LKLR-FUT, MAGE-family, mammaglobin, MAP17, melan-A/MART-1, mesothelin, MIC A/B, MT-MMPs, mucin, MUC-1, NY-ESO-1, osteonectin, p15, P170/MDR1, p53, p97/melanotransferrin, PAI-1, PAP, PDGF, uPA, PRAME, probasin, progenipoientin, PSA, PSM, RAGE-1, Rb, RCAS1, SART-1, SSX-family, STAT3, STn, TAG-72, TGF-alpha, TGF-beta, Thymosin-beta-15, TNF-alpha, TYRP-, TYRP-2, tyrosinase, VEGF, ZAG, p16INK4, and glutathione-S-transferase.

Checkpoint Inhibitors

In certain embodiments, a method of treating cancer, comprises co-culturing ex vivo, CD4⁺ T cells with B-cell activating factor (BAFF)-treated B cells obtained from a subject suffering from cancer and re-infusing the CD4⁺ T cells and BAFF-treated B cells into the subject. The CD4⁺ T cells and BAFF-treated B cells can be co-cultured with tumor cells obtained from the subject or tumor antigens, so as to induce tumor cell specific T cells. The tumor cells can be irradiated. Further, at least one checkpoint inhibitor may be added to the culturing of cells and/or at least one checkpoint inhibitor is co-administered to the patient, during re-infusion of the CD4⁺ T cells and BAFF-treated B cells. In other embodiments, the at least one checkpoint inhibitor is administered to the patient prior to, during and/or after re-infusion of the CD4⁺ T cells and BAFF-treated B cells. Examples of checkpoint inhibitors include without limitation, an inhibitor of: PD-1, PD-L1, PD-L2, CTLA4, TIM-3, LAG-3, CEACAM-1, CEACAM-5, VISTA, BTLA, TIGIT, LAIR1, CD 160, 2B4 or TGFR-β.

The duration and/or dose of treatment with checkpoint inhibitor therapies may vary according to the particular anti-immune checkpoint inhibitor agent or combination thereof (e.g., anti-ARG1 agents like small molecule inhibitors in combination with inhibitors of PD-1, PD-L1, PD-L2, CTLA4, and the like). An appropriate treatment time for a particular cancer therapeutic agent will be appreciated by the skilled artisan. For example, dosage concentrations and dosing regimens are configured based upon one or more cancer related factors such as tumor size, tumor volume, cancer stage of a cancer patient or group of cancer patients (such as pre or post metastatic cancer). The invention contemplates the continued assessment of optimal treatment schedules for each cancer therapeutic agent, where the phenotype of the cancer of the subject as determined by the methods of the invention is a factor in determining optimal treatment doses and schedules.

The Programmed Death 1 (PD-1) protein is an inhibitory member of the extended CD28/CTLA-4 family of T cell regulators (Okazaki et al. (2002) Curr Opin Immunol 14: 391779-82; Bennett et al. (2003) J. Immunol. 170:711-8). Other members of the CD28 family include CD28, CTLA-4, ICOS and BTLA. Two cell surface glycoprotein ligands for PD-1 have been identified, Program Death Ligand 1 (PD-L1) and Program Death Ligand 2 (PD-L2). PD-L1 and PD-L2 have been shown to downregulate T cell activation and cytokine secretion upon binding to PD-1 (Freeman et al. (2000) J Exp Med 192:1027-34; Latchman et al. (2001) Nat Immunol 2:261-8; Carter et al. (2002) Eur J Immunol 32:634-43; Ohigashi et al. (2005) Clin Cancer Res 11:2947-53).

PD-L1 (also known as cluster of differentiation 274 (CD274) or B7 homolog 1 (B7-H1)) is a 40 kDa type 1 transmembrane protein. PD-L1 binds to its receptor, PD-1, found on activated T cells, B cells, and myeloid cells, to modulate activation or inhibition. Both PD-L1 and PD-L2 are B7 homologs that bind to PD-1, but do not bind to CD28 or CTLA-4 (Blank et al. (2005) Cancer Immunol Immunother. 54:307-14). Binding of PD-L1 with its receptor PD-1 on T cells delivers a signal that inhibits TCR-mediated activation of IL-2 production and T cell proliferation. The mechanism involves inhibition of ZAP70 phosphorylation and its association with CD3.zeta. (Sheppard et al. (2004) FEBS Lett. 574:37-41). PD-1 signaling attenuates PKC-0 activation loop phosphorylation resulting from TCR signaling, necessary for the activation of transcription factors NF-κB and AP-1, and for production of IL-2. PD-L1 also binds to the costimulatory molecule CD80 (B7-1), but not CD86 (B7-2) (Butte et al. (2008) Mol Immunol. 45:3567-72).

Expression of PD-L1 on the cell surface has been shown to be upregulated through IFN-γ stimulation. PD-L1 expression has been found in many cancers, including human lung, ovarian and colon carcinoma and various myelomas, and is often associated with poor prognosis (Iwai et al. (2002) PNAS 99:12293-7; Ohigashi et al. (2005) Clin Cancer Res 11:2947-53; Okazaki et al. (2007) Intern. Immun. 19:813-24; Thompson et al. (2006) Cancer Res. 66:3381-5). PD-L1 has been suggested to playa role in tumor immunity by increasing apoptosis of antigen-specific T-cell clones (Dong et al. (2002) Nat Med 8:793-800). It has also been suggested that PD-L1 might be involved in intestinal mucosal inflammation and inhibition of PD-L1 suppresses wasting disease associated with colitis (Kanai et al. (2003) J Immunol 171:4156-63).

Studies with checkpoint inhibitor antibodies for cancer therapy have generated unprecedented response rates in cancers previously thought to be resistant to cancer treatment (see, e.g., Ott & Bhardwaj, 2013, Frontiers in Immunology 4:346; Menzies & Long, 2013, Ther Adv Med Oncol 5:278-85; Pardoll, 2012, Nature Reviews 12:252-264). Therapy with antagonistic checkpoint blocking antibodies against CTLA-4, PD-1 and PD-L1 are one of the most promising new avenues of immunotherapy for cancer and other diseases. In contrast to the majority of anti-cancer agents, checkpoint inhibitor do not target tumor cells directly, but rather target lymphocyte receptors or their ligands in order to enhance the endogenous antitumor activity of the immune system. (Pardoll, 2012, Nature Reviews 12:252-264) Because such antibodies act primarily by regulating the immune response to diseased cells, tissues or pathogens, they may be used in combination with other therapeutic modalities, such as antibody-drug conjugates (ADCs), to enhance the anti-tumor effect of the ADCs.

Programmed cell death protein 1 (PD-1, also known as CD279) encodes a cell surface membrane protein of the immunoglobulin superfamily, which is expressed in B cells and NK cells (Shinohara et al., 1995, Genomics 23:704-6; Blank et al., 2007, Cancer Immunol Immunother 56:739-45; Finger et al., 1997, Gene 197:177-87; Pardoll, 2012, Nature Reviews 12:252-264). Anti-PD1 antibodies have been used for treatment of melanoma, non-small-cell lung cancer, bladder cancer, prostate cancer, colorectal cancer, head and neck cancer, triple-negative breast cancer, leukemia, lymphoma and renal cell cancer (Topalian et al., 2012, N Engl J Med 366:2443-54; Lipson et al., 2013, Clin Cancer Res 19:462-8; Berger et al., 2008, Clin Cancer Res 14:3044-51; Gildener-Leapman et al., 2013, Oral Oncol 49:1089-96; Menzies & Long, 2013, Ther Adv Med Oncol 5:278-85).

Exemplary anti-PD1 antibodies include pembrolizumab (MK-3475, Merck), nivolumab (BMS-936558, Bristol-Myers Squibb), and pidilizumab (CT-011, Curetech LTD.). Anti-PD1 antibodies are commercially available, for example from ABCAM™ (AB137132), BIOLEGEND™ (EH12.2H7, RMP1-14) and Affymetrix Ebioscience (J105, J116, MIH4).

Programmed cell death 1 ligand 1 (PD-L1, also known as CD274) is a ligand for PD-1, found on activated T cells, B cells, myeloid cells and macrophages. The complex of PD-1 and PD-L1 inhibits proliferation of CD8⁺ T cells and reduces the immune response (Topalian et al., 2012, N Engl J Med 366:2443-54; Brahmer et al., 2012, N Eng J Med 366:2455-65). Anti-PDL1 antibodies have been used for treatment of non-small cell lung cancer, melanoma, colorectal cancer, renal-cell cancer, pancreatic cancer, gastric cancer, ovarian cancer, breast cancer, and hematologic malignancies (Brahmer et al., 2012, N Eng J Med 366:2455-65; Ott et al., 2013, Clin Cancer Res 19:5300-9; Radvanyi et al., 2013, Clin Cancer Res 19:5541; Menzies & Long, 2013, Ther Adv Med Oncol 5:278-85; Berger et al., 2008, Clin Cancer Res 14:13044-51).

Exemplary anti-PDL1 antibodies include MDX-1105 (MEDAREX), durvalumab (MEDI4736, MEDIMMUNE) atezolizumab (TECENTRIQ™, MPDL3280A, GENENTECH) and BMS-936559 (BRISTOL-MYERS SQUIBB). Anti-PDL1 antibodies are also commercially available, for example from AFFYMETRIX EBIOSCIENCE (MIH1).

In certain embodiments, a checkpoint inhibitor is an RNA interfering agent.

Combination Therapies.

The compositions of the invention embodied herein, can be administered with one or more alternative treatment regimens, such as targeted and/or untargeted anti-cancer therapies can be administered. Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with or without anti-immune checkpoint inhibitor therapy.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine: DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-L and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34; 3-aminobenzamide (Trevigen); 4-amino-1,8-naphthalimide; (Trevigen); 6(5H)-phenanthridinone (Trevigen); benzamide (U.S. Pat. Re. 36,397); and NU1025. The mechanism of action is generally related to the ability of PARP inhibitors to bind PARP and decrease its activity. PARP catalyzes the conversion of .beta.-nicotinamide adenine dinucleotide (NAD⁺) into nicotinamide and poly-ADP-ribose (PAR). Both poly (ADP-ribose) and PARP have been linked to regulation of transcription, cell proliferation, genomic stability, and carcinogenesis (Bouchard V. J. et al., Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q. et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125 palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In another embodiment, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water, implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer; by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

Pharmaceutical Compositions

The compositions of the invention can be administered as pharmaceutical compositions. In certain embodiments, a pharmaceutical composition comprises an isolated cell wherein the isolated cell comprises a vector encoding a B-cell activating factor (BAFF) or active fragments thereof. In certain embodiments, the pharmaceutical composition further comprises a checkpoint inhibitor. In certain embodiments, the checkpoint inhibitor comprises an inhibitor of: PD-1, PD-L1, PD-L2, CTLA4, TIM-3, LAG-3, CEACAM-1, CEACAM-5, VISTA, BTLA, TIGIT, LAIR1, CD 160, 2B4 or TGFR-β. The isolated cell is a B cell, a stem cell, a bone marrow cell, a tumor cell, or cell-line.

The pharmaceutical compositions of the present invention may be specially formulated, in pharmaceutically acceptable carriers or salts, for parenteral administration, for example, by subcutaneous, intramuscular or intravenous injection as, for example, a sterile solution or suspension; intravaginally or intrarectally, for example, as a pessary, cream or foam; or aerosol, for example, as an aqueous aerosol, liposomal preparation or solid particles containing the compound.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose, (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar, (14) buffering agents, such as magnesium hydroxide and aluminum hydroxide; (15) alginic acid; (16) pyrogen-free water, (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity, or expression and/or activity of the complex encompassed by the invention. These salts can be prepared in situ during the final isolation and purification of the agents, or by separately reacting a purified agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression and/or activity, or expression and/or activity of the complex. These salts can likewise be prepared in situ during the final isolation and purification of the agents, or by separately reacting the purified agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Example 1: B Cells Treated with BAFF Show High Levels of PD-L1 Expression

1×10⁶ splenocytes from a B6 mouse were cultured for 72 hours in a flat bottom well of a 48-well plate containing 400 microliters of CLT medium (RPMI with 10% Fetal bovine serum, 1% L-glutamine, 1% penicillin/streptomycin, and 50 μM β-mercaptoethanol). Recombinant mouse BAFF (0 μg, 1 μg, or 2 μg) was added to each well.

Flow cytometry was performed on the cells after 72 hours. The BAFF-treated cells express high levels of PD-L1 in a dose-dependent fashion.

### Example 2: B Cells Treated with BAFF Show Increased CD86 and CD40 Expression

As compared to control, BAFF-cultured B cells show increased expression of CD86 and CD40. As compared to B cells activated by ant-IgM antibody, B cells activated by BAFF show increased expression of PD-L1 and decreased expression of PD-1.

### Example 3: BAFF-Treated B Cells have an Improved Ability to Stimulate CD4⁺ T Cells

B cells were isolated from a B6 mouse using a EASYSEP™ Human B Cell Enrichment Kit. 0.5×10⁶ B cells per well were cultured in a flat bottom well of a 48-well plate containing 400 microliters of CLT medium (RPMI with 10% Fetal bovine serum, 1% L-glutamine, 1% penicillin/streptomycin, and 50 μM β-mercaptoethanol). Control or recombinant mouse BAFF (3 μg) were added to the well for 24 hours. For the last 8 hours, Ova peptide (sequence SLKISQAVHAAHAEINEAGR (SEQ ID NO: 1)) were added. The plate was then spun and washed three times to remove any BAFF or excess peptide not processed by the B cells. B cells were resuspended in 400 microliters of fresh CLT medium. 1×10⁶ CD4 T cells from an OT2 mouse were added to each well containing B cells, allowing 48 hours of co-culture with the B cells.

CD4 T cells co-cultured with BAFF-treated peptide pulsed B cells showed significant increase in CD44 and CD69 expression at the end of the 48 hour co-culture experiment.

This experiment was repeated with CFSE-stained CD4 T cells to demonstrate CD4 T cell proliferation.

### Example 4: BAFF is an Effective Therapeutic Anticancer Vaccine Adjuvant

A 3T3-derived cell line was created that secretes mouse BAFF. Here it was shown that use of this bystander BAFF-secreting cell line induced a more potent anti-tumor immune response when co-administered with irradiated tumor cells, as compared to a 3T3-mock cell line which does not secrete BAFF. 20 NeuN mice were administered 5×10⁵ NT2.5 tumor cells (a mouse model of HER2⁺ breast cancer), injected subcutaneously into the R mammillary gland. On day 3 after tumor inoculation, mice received 3×10⁶ irradiated 3T3-BAFF+3×10⁶ irradiated NT2.5 tumor cells (BAFF-Vac, n=10) or 3×10⁶ irradiated 3T3-mock+3×10⁶ irradiated NT2.5 tumor cells (Mock-Vac, n=10), injected over 3 limbs. Survival for the group receiving irradiated 3T3-BAFF+NT2.5 tumor cells was greater than the group receiving the mock vaccine.

### Example 5: BAFF as a Vaccine Adjuvant can Increase the Number of Anti-Tumor IgG Antibodies

Mice were treated as described above in Example 4. At days 7 and 15 after vaccination with BAFF-Vac or Mock-Vac, 150 μl were collected using tail bleeds from each mouse using heparinized capillary tubes. The collected blood was spun down and serum was collected. Using FACs of the NeuN tumor cells, with the mouse serum used as a primary antibody, and using a secondary anti-IgG antibody, a marked increase in anti-tumor IgG was evident at 15 days.

### Example 6: B Cells Treated with BAFF Show High Levels of PD-L1 and MHCII Expression

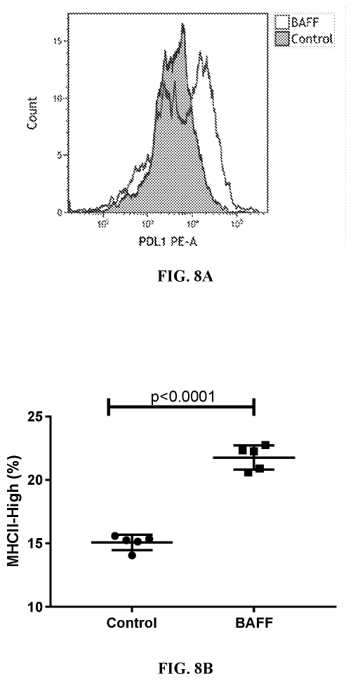

1×10⁶ splenocytes from a B6 mouse were cultured for 72 hours in a flat bottom well of a 48-well plate containing 400 microliters of CLT medium (RPMI with 10% Fetal bovine serum, 1% L-glutamine, 1% penicillin/streptomycin, and 50 μM β-mercaptoethanol), with or without recombinant mouse BAFF (1 μg). Flow cytometry was performed on the cells after 72 hours. The BAFF-treated cells express high levels of PD-L1 (FIG. 8A) in a dose-dependent fashion.

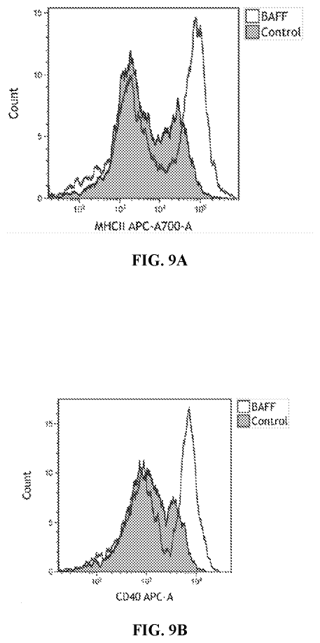

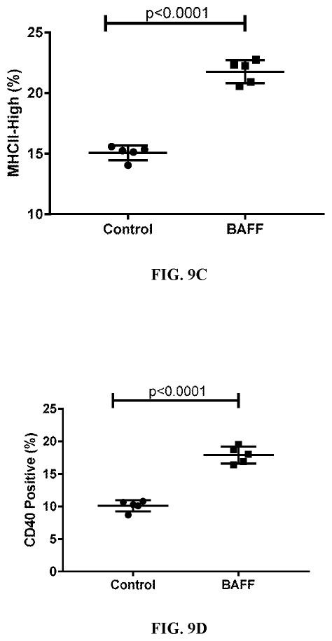

B cells treated with BAFF show increased MHCII (FIGS. 9A and 9C) and CD40 (FIGS. 9B and 9D) expression.

### Example 7: BAFF-Treated B Cells have an Improved Ability to Stimulate CD4⁺ T Cells

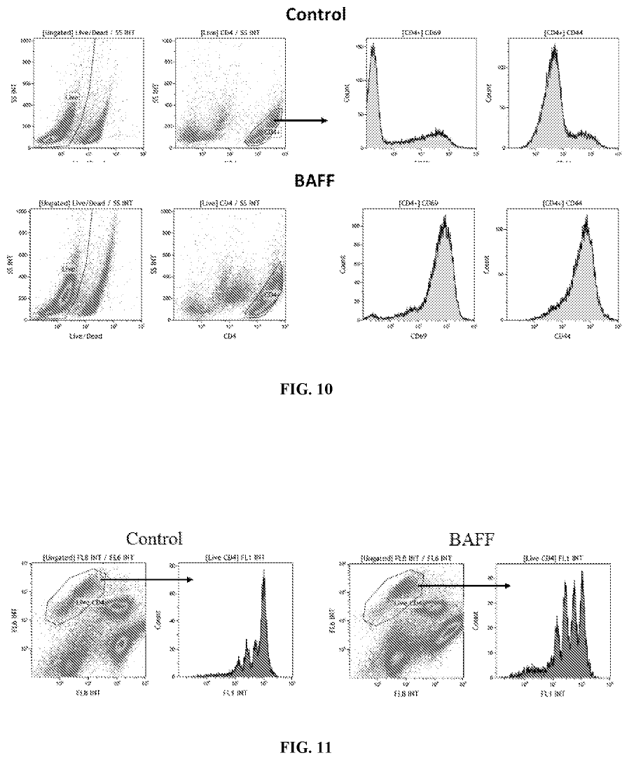

B cells were isolated from a B6 mouse using an EASYSEP™ Human B Cell Enrichment Kit. 0.5×10⁶ B cells per well were cultured in a flat bottom well of a 48-well plate containing 400 microliters of CLT medium (RPMI with 10% Fetal bovine serum, 1% L-glutamine, 1% penicillin/streptomycin, and 50 μM β-mercaptoethanol). Control or recombinant mouse BAFF (3 μg) were added to the well for 24 hours. For the last 8 hours, Ova peptide (sequence SLKISQAVHAAHAEINEAGR, SEQ ID NO: 1) were added. The plate was then spun and washed three times to remove any BAFF or excess peptide not processed by the B cells. B cells were resuspended in 400 microliters of fresh CLT medium. 1×10⁶ CD4 T cells from an OT2 mouse were added to each well containing B cells, allowing 48 hours of coculture with the B cells. CD4 T cells cocultured with BAFF-treated peptide pulsed B cells showed significant increase in CD44 and CD69 expression at the end of the 48 hour coculture experiment (FIG. 10).

### Example 8: BAFF-Treated B Cells have an Improved Ability to Stimulate CD4⁺ T Cells

The experiments from FIG. 10 were repeated using CFSE-stained CD4⁺ T cells to demonstrate CD4⁺ T cell proliferation. These results show that BAFF-treated B cells have increased activation and an improved ability to stimulate and expand CD4⁺ T cells. However, they upregulate PD-L1 as a major compensatory mechanism.

### Example 9: Systemic Recombinant BAFF Administration

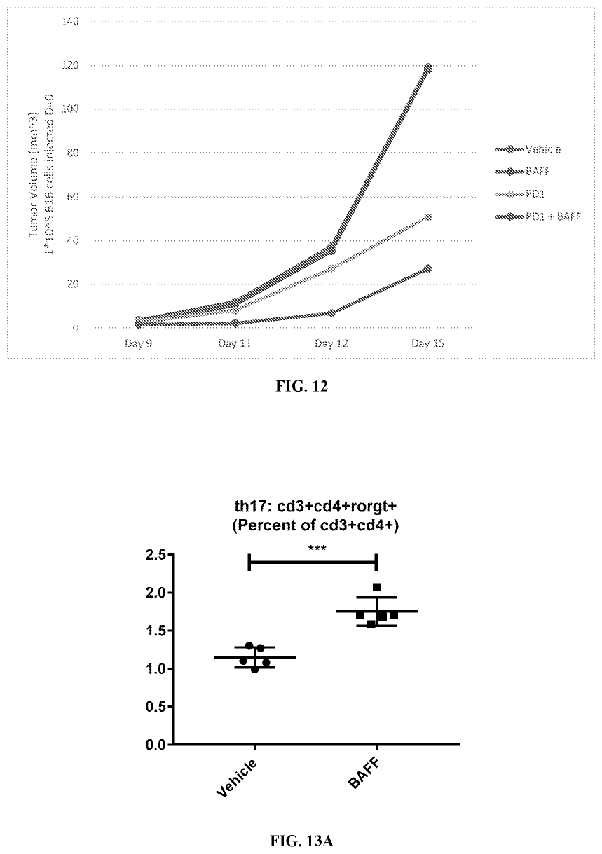

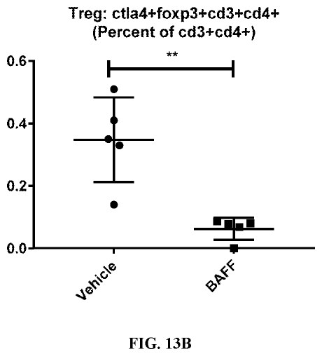

Systemic recombinant BAFF administration synergizes with anti-PD-1 therapy to promote anti-tumor immunity (FIG. 12). FIGS. 13A and 13B demonstrate that systemic recombinant BAFF reduces T regulatory cells (FIG. 13B) and increased TH17 (FIG. 13A) cells in tumor bearing mice.

### Example 10: BAFF is an Effective Therapeutic Anticancer Vaccine Adjuvant

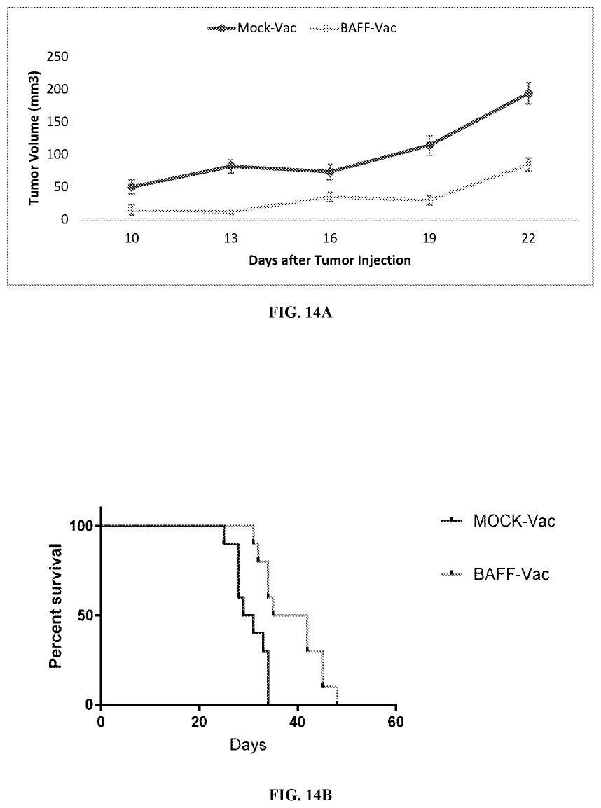

Results shown in FIGS. 14A and 14B demonstrate that BAFF is an effective therapeutic anticancer vaccine adjuvant. FIG. 14A compares the tumor volume (mm³) between Mock-Vac and BAFF-Vac. FIG. 14B compares the percent survival between mice receiving the Mock-Vac versus the BAFF-Vac. A 3T3-derived cell line that secretes mouse BAFF was created. Use of this bystander BAFF-secreting cell line induces a more potent anti-tumor immune response when coadministered with irradiated tumor cells, as compared to a 3T3-mock cell line which does not secrete BAFF. Here, 20 NeuN mice were administered 5×10⁵ NT2.5 tumor cells (a mouse model of HER2⁺ breast cancer), injected subcutaneously into the R mammillary gland. On day 3 after tumor inoculation, mice received 3×10⁶ irradiated 3T3-BAFF+3×10⁶ irradiated NT2.5 tumor cells (BAFF-Vac, n=10) or 3×10⁶ irradiated 3T3-mock+3×10⁶ irradiated NT2.5 tumor cells (Mock-Vac, n=10), injected over 3 limbs. Survival for the group receiving irradiated 3T3-BAFF+NT2.5 tumor cells was greater than the group receiving the mock vaccine.

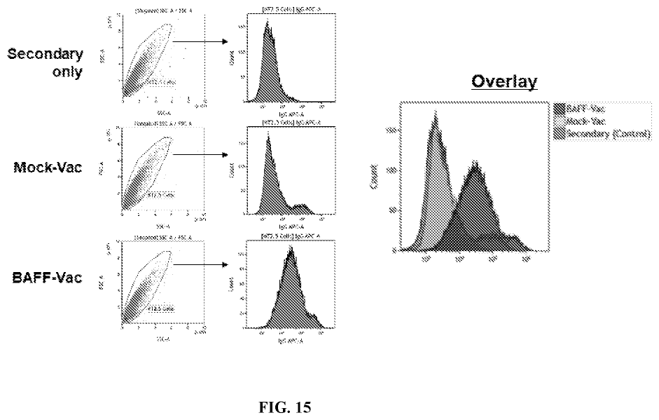

BAFF as a vaccine adjuvant can also increase the number of anti-tumor IgG antibodies (FIG. 15). Mice were treated as described above. At days 7 and 15 after vaccination with BAFF-Vac or Mock-Vac, 150 μl was collected using tail bleeds from each mouse using heparinized capillary tubes. The collected blood was spun down and serum was collected. Using FACs of the NeuN tumor cells, with the mouse serum used as a primary antibody, and using a secondary anti-IgG antibody, a marked increase in anti-tumor IgG was found at 15 days.

### OTHER EMBODIMENTS

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. A method of treating cancer comprising:
administering to a subject in need thereof, a composition comprising a therapeutically effective amount of B-cell activating factor (BAFF) wherein the BAFF activates an immune response to a tumor;
thereby treating cancer.

2. The method of claim 1, wherein further comprising administering a tumor-specific antigen.

3. The method of claim 1, wherein the BAFF increases B-cell expression of CD40 and/or CD86 as compared to a control.

4. The method of claim 1, wherein the BAFF is protein or active fragment thereof.

5. A method of increasing an antigen specific immune response in vivo, comprising administering to a subject a composition comprising BAFF and a desired antigen for which a specific immune response is desired.

6. The method of claim 5, wherein the antigen comprises: a vaccine, a peptide, an irradiated cell, polynucleotide, oligonucleotide or combinations thereof.

7. The method of claim 5 wherein the antigen is a tumor antigen, a virus antigen or a combination thereof.

8. (canceled)

9. The method of claim 50, wherein the at least one checkpoint inhibitor comprises an inhibitor of: PD-1, PD-L1, PD-L2, CTLA4, TIM-3, LAG-3, CEACAM-1, CEACAM-5, VISTA, BTLA, TIGIT, LAIR1, CD 160, 2B4 or TGFR-β.

10. The method of claim 50, wherein the at least one checkpoint inhibitor is anti-PD-1 antibody.

11. The method of claim 50, wherein the BAFF increases B-cell expression of CD40 and/or CD86 as compared to a control.

12. The method of claim 50, wherein BAFF-treated B cells stimulate CD4⁺ T cell activity.

13. The method of claim 50, wherein the BAFF increases reduces T regulatory cell activity and increases TH17 activity.

14. The method of claim 50, wherein the BAFF is protein or active fragment thereof.

15. A method of treating cancer, comprising:
co-culturing ex vivo, CD4⁺ T cells with B-cell activating factor (BAFF)-treated B cells obtained from a subject suffering from cancer;
re-infusing the CD4⁺ T cells and BAFF-treated B cells into the subject;
thereby treating the subject's cancer.

16. The method of claim 15, wherein the CD4⁺ T cells and BAFF-treated B cells are co-cultured with tumor cells obtained from the subject.

17. The method of claim 15, wherein the CD4⁺ T cells and BAFF-treated B cells are co-cultured with tumor cell antigens.

18. The method of claim 15, further comprising co-culturing the CD4⁺ T cells and BAFF-treated B cells with at least one checkpoint inhibitor.

19. The method of claim 15, wherein the at least one checkpoint inhibitor is co-administered to the patient, during re-infusion of the CD4⁺ T cells and BAFF-treated B cells.

20. The method of claim 18, wherein the at least one checkpoint inhibitor is administered to the patient prior to, during and/or after re-infusion of the CD4⁺ T cells and BAFF-treated B cells.

21. The method of claim 18, wherein the at least one checkpoint inhibitor comprises an inhibitor of: PD-1, PD-L1, PD-L2, CTLA4, TIM-3, LAG-3, CEACAM-1, CEACAM-5, VISTA, BTLA, TIGIT, LAIR1, CD 160, 2B4 or TGFR-β.

22-49. (canceled)

50. The method of claim 1 comprising administering to the subject a composition comprising a therapeutically effective amount of B-cell activating factor (BAFF) and at least one checkpoint inhibitor;
